(19) United States
(12) Patent Application Publication
(10) Pub. No.: US 2005/0070939 A1
(43) Pub. Date: Mar. 31, 2005

(54) Unfolding anastomosis ring device

(76) Inventor: Jean Beaupre, Cincinnati, OH (US)

Correspondence Address: FROST BROWN TODD, LLC, 2200 PNC CENTER, 201 E. FIFTH STREET, CINCINNATI, OH, 45202, US

(21) Appl. No.: 10/675,091

(22) Filed: Sep. 30, 2003

(51) Int. Cl. A61B 17/08

(52) U.S. Cl. 606/154

(57) ABSTRACT

An anastomotic ring device for forming a hollow rivet (ring) attachment between tissue lumens facilitates laparoscopic or endoscopic implantation by including features that facilitate assembly, specifically by forming from constituent arcuate members or petals that join with one another at a circular midpoint. Automated assembly is thereby facilitated with each longitudinal half including a “crown” of these arcuate members arranged in a cylindrical pattern with equally spaced curved points and overlapping ends. Molded arcuate members with integral hinges further enhance assembly and provide other advantages, especially when implanted with an applier capable of actuating the anastomotic ring device.

### CROSS REFERENCE TO RELATED APPLICATIONS

The present application is related to five co-pending and commonly-owned application filed on even date herewith, the disclosure of each is hereby incorporated by reference in its entirety:
“Anastomosis Wire Ring Device”, Ser. No. ______ to Don Tanaka, Mark Ortiz and Darrell Powell;
“Applier For Fastener For Single Lumen Access Anastomosis”, Ser. No. ______ to Mark Ortiz;
“Single Lumen Access Deployable Ring for Intralumenal Anastomosis”, Ser. No. ______ to Mark Ortiz; and
“Single Lumen Anastamosis Applier for Fastener”, Ser. No. ______ to Mark Ortiz, Robert McKenna, Bill Kraimer, Mike Stokes, and Foster Stulen.

### FIELD OF THE INVENTION

The present invention relates, in general, to devices and methods for surgically modifying organs and vessels. More particularly, it relates to anastomosis devices for joining two organs such as, for example, two separate lengths of small bowel to each other, a section of small bowel to the stomach, or the common bile duct to the duodenum in a procedure called a choledochoduodenostomy. Vascular anastomosis could be performed as well.

### BACKGROUND OF THE INVENTION

Creating an anastomosis, or the surgical formation of a passage between two normally distinct vessels, is a critical step of many surgical procedures. This is particularly true of gastric bypass procedures in which two portions of small intestine are joined together and another portion of small intestine is joined to the stomach of the patient. This is also true of surgery to alleviate blockage in the common bile duct by draining bile from the duct to the small intestine during surgery for pancreatic cancer.

The percentage of the world population suffering from morbid obesity is steadily increasing. Severely obese persons are susceptible to increased risk of heart disease, stroke, diabetes, pulmonary disease, and accidents. Because of the effect of morbid obesity to the life of the patient, methods of treating morbid obesity are being researched.

Numerous non-operative therapies for morbid obesity have been tried with virtually no permanent success. Dietary counseling, behavior modification, wiring a patient's jaws shut, and pharmacologic methods have all been tried, and though temporarily effective, failed to correct the condition. Further, introducing an object in the stomach, such as an esophago-gastric balloon, to fill the stomach have also been used to treat the condition; however, such approaches tend to cause irritation to the stomach and are not effective long-term.

Surgical treatments of morbid obesity have been increasingly used with greater success. These approaches may be generalized as those that reduce the effective size of the stomach, limiting the amount of food intake, and those that create malabsorption of the food that it is eaten. For instance, some patients benefit from adjustable gastric bands (AGB) that are advantageously laparoscopically placed about the stomach to form a stoma of a desired size that allows food to fill an upper portion of the stomach, causing a feeling of satiety. To allow adjustment of the size of the stoma after implantation, a fluid conduit communicates between an inwardly presented fluid bladder of the AGB to a fluid injection port subcutaneously placed in front of the patient's sternum. A syringe needle may then inject or withdraw fluid as desired to adjust the AGB.

Although an effective approach to obesity for some, other patients may find the lifestyle changes undesirable, necessitated by the restricted amount of food intake. In addition, the medical condition of the patient may suggest the need for a more permanent solution. To that end, surgical approaches have been used to alter the portions of the stomach and/or small intestine available for digesting food. Current methods of performing a laparoscopic anastomoses for a gastric bypass include stapling, suturing, and placing biofragmentable rings, each having significant challenges. For instance, suturing is time consuming, as well as being technique and dexterity dependent. Stapling requires placement of an anvil, which is a large device that cannot be introduced through a trocar port. Having to introduce the port through a laparotomy presents an increased incidence of wound site infection associated with intralumenal content being dragged to the laparotomy entry site.

As an example of the latter approach, in U.S. Pat. No. 6,543,456 a method for gastric bypass surgery includes the insertion of proximal and distal anastomosis members (e.g., anvils) transorally with grasping forceps. The stomach and the small intestine are transected endoscopically by a surgical severing and stapling instrument to create a gastric pouch, a drainage loop, and a Roux limb. An endoscopically inserted circular stapler attaches to the distal anastomosis member to join the drainage loop to a distal portion of the intestine, and the circular stapler attaches to the proximal anastomosis member to join the Roux limb to the gastric pouch. Thereafter, the anastomosis members are removed to create an orifice between joined portions of the stomach and intestine. This method reduces the number of laparoscopic ports, avoids a laparoscopic insertion of an anastomosis instrument (e.g., circular stapler) into an enlarged surgical port, and eliminates the need for an enterotomy and an enterotomy closure.

For many anastomoses, surgeons use circular staplers, linear staplers, or manual sutures. However, to reduce incision size and to make the surgical process less technically demanding and time consuming, an anastomotic device that deforms to hold tissue portions together when the device is ejected from a constraining enclosure has been described. Such an approach is described in U.S. Pat. Appl. Publ. No. US 2003/0032967 and PCT application WO 03/000142 both to Adrian Park et al, which is hereby incorporated herein by reference, describes such a device. Therein, gastrointestinal or enteric (including biliary) anastomosis is achieved by insertion of a sheath that perforates the walls of two tissue passages, such as the stomach and small intestine. A three-dimensional woven tube of wire of having a thermal shape memory effect (SME) (“generally-known nitinol ring device”) is presented by a cannula of the sheath on both sides of the openings. Deployment of the woven tube causes the outer loops or ends of the tube to fold or loop back to hold the luminal interface of the anastomosis site in apposition. Thereby, the need for a mechanical compression component in a delivery system is reduced or avoided, reducing the size and complexity of the delivery device.

The anastomotic device disclosed in WO 03/000142 is constrained by a retractable sheath to an advantageous small-diameter tubular shape. A surgeon applies the anastomotic device by maneuvering the sheath through the tissue portions requiring anastomosis and retracting the sheath. Retracting the sheath removes the constraint on the device, allowing the device to assume a roughly hourglass shape. The larger ends of the hourglass shape hold the two tissue portions together in an effective anastomosis.

The constrained anastomotic device, which may be made of a shape memory material such as nitinol, exerts a force against the inner diameter of the sheath and tends to warp towards its roughly hourglass-shaped deployed position. When the sheath is retracted proximally, the forces generated by the device in transition from a tubular shape to an hourglass shape urge the anastomotic device distally. This device movement makes surgical control harder to achieve when placing the device through the otomies of two tissue portions requiring anastomosis.

While the generally-known nitinol ring device is a significant advancement in the treatment of morbid obesity, it is believed that further improvements would be desirable. For instance, the continuous interlocking petals are difficult to manufacturer, especially since the depicted woven tube is of a continuous wire loop bent into a pattern of interlocking triangles that are hand woven from two wire strands and the four free ends connected to one another.

Consequently, there is a general need for an approach to making an anastomosis ring device that can be used in existing trocar ports (e.g., 12 mm size) and that reliably and effectively creates an anastomotic attachment between lumens, eliminate the need for surgical stapling and suturing to form an anastomosis.

### BRIEF SUMMARY OF THE INVENTION

The invention overcomes the above-noted and other deficiencies of the prior art by providing a method of making an anastomotic ring device of interlocking sinusoidal members in a cylindrical shape (undeployed) that readily converts to a hollow rivet shape (deployed), or “hour glass shape”, for endoscopic surgical procedures. In particular, each anastomotic ring device is assembled from a plurality of “points”, or arcuate members. These components allow for a proximal and distal longitudinal halves (i.e., “crowns”) of the ring device to be assembled individually and joined together, which lends itself for simplified manufacture.

In one aspect of the invention, a longitudinally bisected ring device is assembled from arcuate members of a deformable material (e.g., nitinol or other alloy). Each arcuate member is of identical or similar points (e.g., two diverging legs joined at an acutely bent radius). One crown is formed by placing half of the arcuate members (e.g., 10) in cylindrical configuration, overlapping each leg with the leg of an adjacent arcuate member. The crown may be held in a fixture until mated to another crown that is inverted to the first. The longitudinally bisected ring device thus formed in its undeployed, cylindrical shape may receive further processing to impart an ability to actuate to a hollow rivet shape to hold two tissue lumens at an anastomotic attachment.

In another aspect of the invention, such a method of implanting the longitudinally bisected ring device includes not having to rely solely or at all upon an intrinsic actuation potential. Instead, an actuator member of an applier is capable of receiving the cylindrical, undeployed shape of the ring device. When inserted across the anastomotic attachment site, the applier actuates the actuator member by compressing the ring device into a hollow rivet shape.

In yet another aspect of the invention, a longitudinally bisected ring device is formed from molded arcuate members assembled into interlocking cylindrical sinusoids that hingedly attach with adjacent arcuate members in their same longitudinal half of the ring device and rigidly attach to inverted arcuate members in the other longitudinal half. These molded arcuate members advantageously lend themselves to assembly in a deployed configuration. The ability for the interlocking sinusoids to hinge, allowing overlapping petals to move between cylindrical orientation and hollow rivet shape, lend themselves to implantation at an anastomotic surgical site, such as with the afore-described applier.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain the principles of the present invention.

FIG. 1 is perspective view of an anastomotic ring device assembled from a plurality of arcuate petals.

FIG. 2 is a perspective view of one longitudinal half, or “crown” of the anastomotic ring device of FIG. 1 being assembled onto a fixture.

FIG. 3 is a perspective view of the two halves, or a crown and mirrored or inverted crown prior to attachment one to the other.

FIG. 4 is a perspective view of an applier capable of implantation, actuation and deployment of an anastomotic ring device of FIG. 1, which is retained in an unactuated, cylindrical shape.

FIG. 5 is a detail view of the distal tip including an actuating member and piercing tip of the applier of FIG. 4 retaining the anastomotic ring device of FIG. 1 by gripping each point of each respective petal.

FIG. 6 is a perspective the applier of FIG. 4 actuating with opposing, compressive longitudinal actuation motions the anastomotic ring device of FIG. 1 into an actuated hollow rivet, or hour glass, shape to form an anastomotic attachment.

FIG. 7 is a detail view of the distal tip of the applier of FIG. 6, depicting the actuated anastomotic ring device having been released from the actuating member in preparation for deployment from the applier.

FIG. 8 is a perspective view of hinged anastomotic ring device assembled in an actuated condition from a plurality of molded arcuate petals.

FIG. 9 is a perspective view of one molded arcuate petal.

FIG. 10 is a perspective view of one half of the hinged anastomotic ring device of FIG. 8 assembled into hinged petals.

FIG. 11 is a perspective view of the applier of FIG. 4 actuating to implant the hinged anastomotic ring device of FIG. 8.

FIG. 12 is a detail view of the distal tip of the applier of FIG. 11 depicting the actuated hinged anastomotic ring device having been released from the actuating member in preparation for deployment from the applier.

### DETAILED DESCRIPTION OF THE INVENTION

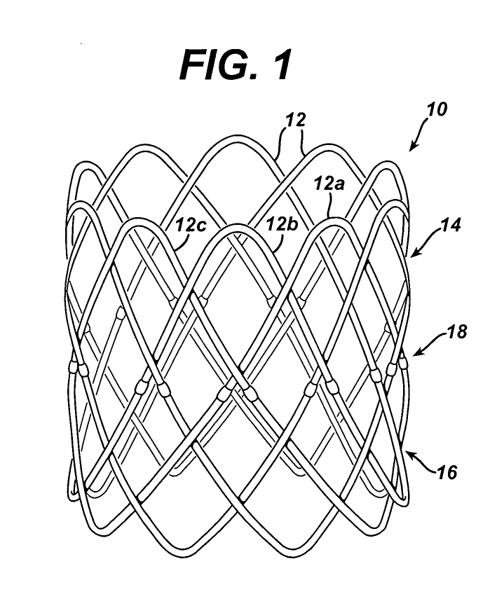

Turning to the Drawings, wherein like numerals denote like components throughout the several views, FIG. 1 depicts an anastomotic ring device 10 in its generally cylindrical, unactuated condition, with its woven tube of strands resembling the interweaving of a chain link fence. In the illustrative embodiment, a plurality of arcuate members, or petals, 12 are assembled in a longitudinal half, or crown 14, intended to be on one side of an anastomotic attachment site, with a similar but inverted or mirrored crown 16, intended to be on the other side of the anastomotic attachment site.

Both halves or crowns 14, 16 are attached at a circular midpoint 18 such that the plurality of arcuate members 12 resemble a plurality woven sinusoids. At the midpoint 18, attachments 20 respectively between pair of end 22, 24 from an arcuate member 12 in the top crown 14 is made to a respective end 22, 24 of an arcuate member 12 in the bottom crown 16. A non-exclusive list of couplings 20 include snap fits, glue, ultrasonic welding, thermal adhesives, etc.

In the illustrative ring device of FIG. 1, ten arcuate members 12 are included in each crown 14, 16. Each arcuate member is woven with two arcuate members to each side and attached to two arcuate members in the other half. For instance, for arcuate member 12a, a left end 22a passes in front (outside) of a right end 24b of a left adjacent arcuate member 12b and then passing behind a right end 24c of a farther left arcuate member 12c, with the pattern repeated about the circumference of the crown 14. It should be appreciated that this number of arcuate members and this degree of interweaving is illustrative and that other patterns consistent with aspects of the invention may be used.

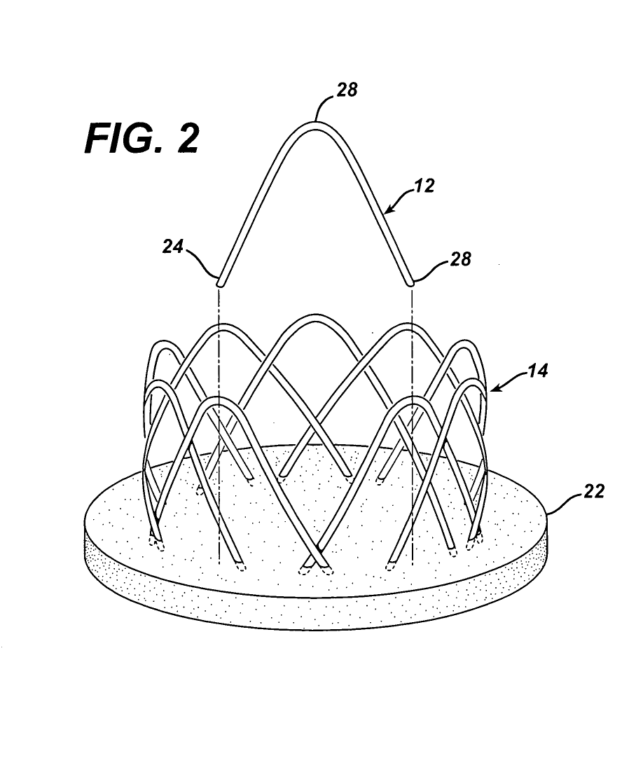

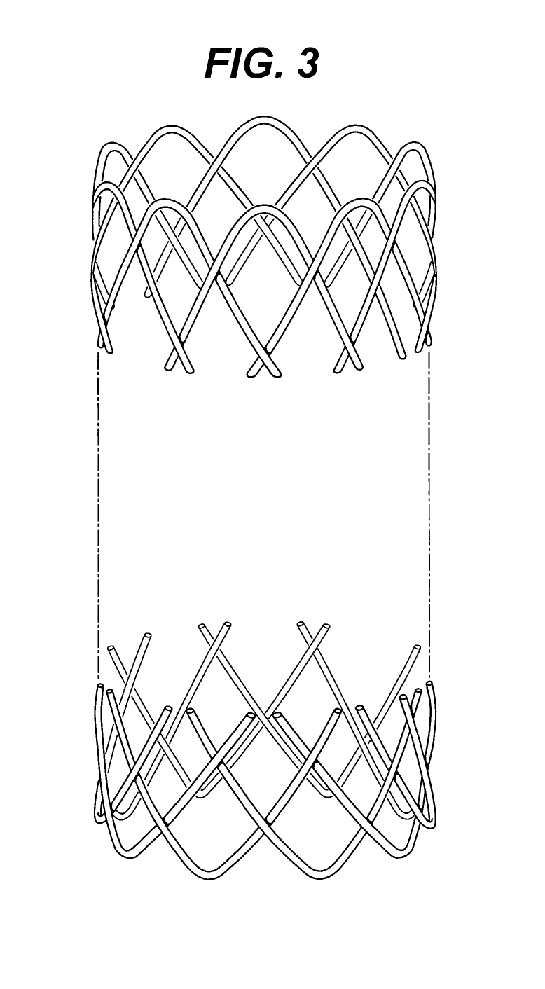

In FIG. 2, assembly of the crown 14 is depicted, illustrating how the plurality of arcuate members 12 facilitates economical manufacture that may be performed by automated mechanisms. In this depiction, a fixture, or disk, 26 holds the plurality of arcuate members 12 until the crown 14 is complete, specifically locating each pair of ends 22, 24 of each arcuate member 12 for attachment to the other crown 16 (shown in FIG. 3). Moreover, the fixture 26, ensures that each curved point 28, from which each end 22, 24 diverges, is equidistantly spaced about the crown 14 and evenly extending for engagement by an applier.

Anastomotic Ring Device Applier.

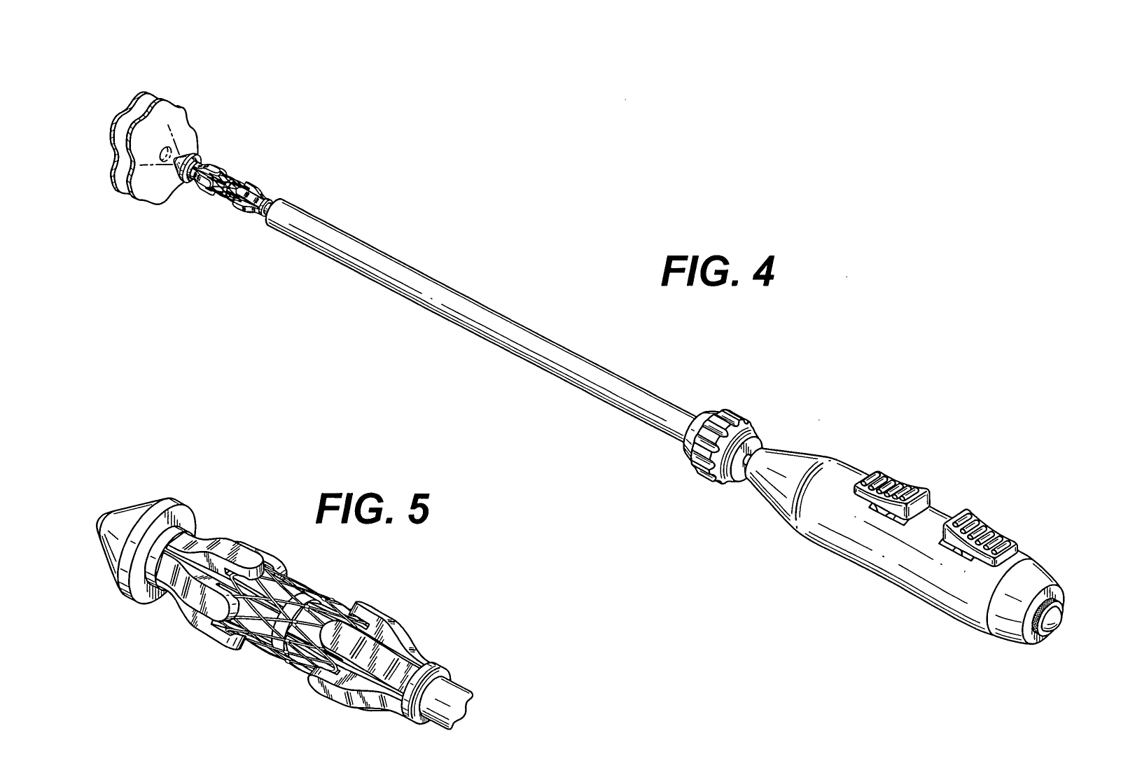

In FIGS. 4-7, an illustrative applier 30 has the anastomotic ring device 12 advantageously retained in a generally cylindrical shape (FIGS. 4-5) distal to an outer tube 32 upon a molded actuation member 34 forming a cannula 36 that distally terminates in a flared tip 38. This flared tip 38 presents a distal piercing surface 40 to form an anastomotic opening 42 through apposite tissue walls 44, 46 of two gastrointestinal passages.

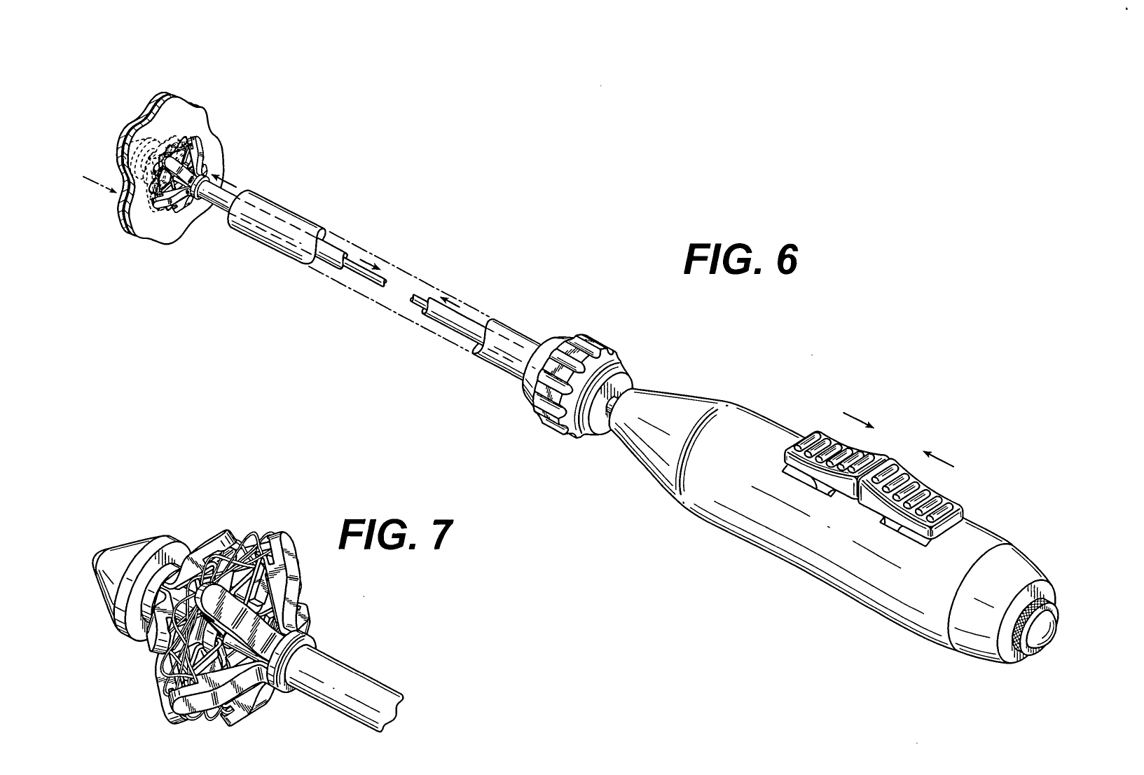

With particular reference to FIG. 6, a handle 48, proximal to the cannula 36, includes a pair of longitudinally aligned triggers 50, 52. The proximal trigger 50, shown at its most proximal, unfired position, is coupled to proximal leaves 54 of the molded actuation member 34 via an intermediate tube 56 of the cannula 36. Distal movement of the proximal trigger 50 thus causes longitudinal distal movement of the intermediate tube 56 and proximal leaves 54, which outwardly actuate like an umbrella by a hinged relationship to a central portion 58 of the molded actuation member 34. (Unlike an umbrella, the “top” is brought toward the center rather than the converse.)

Similarly, the distal trigger 42, shown at its most distal, unfired position, is coupled to distal leaves 60 of the molded actuation member 34 via an internal rod 62 that is coupled for movement within the intermediate tube 56. Proximal movement of the distal trigger 38 causes longitudinal proximal movement of the rod 62 and distal leaves 64 of the molded actuation member 34, which outwardly actuate by a hinged relationship to the central portion 58.

In FIGS. 6-7, the triggers 50, 52 have been slid toward one another to actuate the molded actuating member 34. Specifically, the distal trigger 52 has been moved proximally, causing similar distal movement of the internal rod 62, the distal terminating end of the latter being attached to flared tip 38. The flared tip 38 thus moves toward the distal end of the intermediate tube 56. The proximal trigger 50 has been moved distally, moving intermediate tube 56 also distally. The molded actuating member 34 is compressed between the inwardly moving flared tip 38 and intermediate tube 56. The distal leaves 64 actuate lateral to the longitudinal axis, and move toward and interdigitate with the proximal leaves 54. This movement expedites actuating of an anastomotic ring device 10.

In use, the flared tip 24 of the applier 30 is inserted through a trocar port into a tissue passage that has been placed proximate to another tissue passage that are to be anastomotically joined (See FIGS. 4-5). The flared tip 38 and a distal half of the molded actuating member 34 and anastomotic ring device 12 are inserted through an anastomotic opening 42 formed therebetween and then the applier 30 is actuated. With particular reference to FIGS. 6-7, the proximal and distal leaves 54, 64 are shown as having gripping slots 66 that grip respective curved points 28 of each arcuate member or petals 12 of the anastomotic ring device 12, especially in its unactuated, generally cylindrical shape. These gripping slots 66 assist in preventing the anastomotic ring device 12 from slipping off of the applier 30 or being inappropriately placed thereon for actuation until fully actuated, forming the anastomotic ring device 12 into a hollow rivet shape or hourglass shape to form the anastomotic attachment between tissue walls 44, 46. The fully actuated proximal and distal leaves 54, 64 cause the curved points 28 to disengage from the gripping slots 66. Thereafter, the applier 30 is returned to an unactuated condition and the actuated anastomotic ring device 12 deployed by withdrawing the flared tip 38 from the anastomotic opening 42 and ring device 12.

It should be appreciated that the unactuated anastomotic ring device 10 may be formed from nitinol and temperature treated to create a Shape Memory Effect that would cause self-actuation after implantation to a hollow rivet or hourglass shape, thus allowing generally known appliers to be used. However, as described above and in more detail in the above-referenced co-pending application entitled “Single Lumen Anastamosis Applier for Self-Deploying Fastener” to M. Ortiz, such actuation is enhanced or performed entirely by the applier 30 capable of causing the rapid actuation of the anastomotic ring device 10, thus allowing other materials to be used as well as nitinol. Moreover, the ability to cause actuation with an applier 30 enables the use of ring devices with no inherent actuating ability.

Hinged Anastomotic Ring Device.

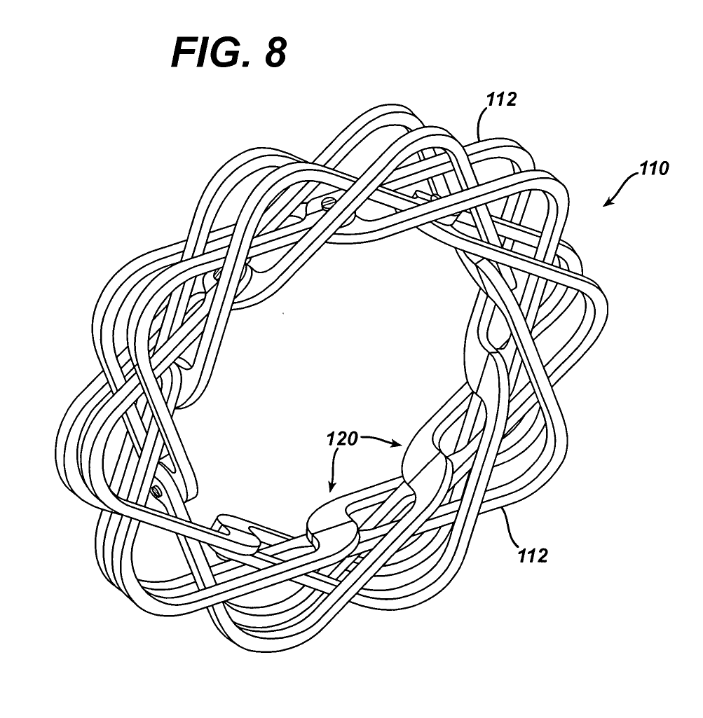

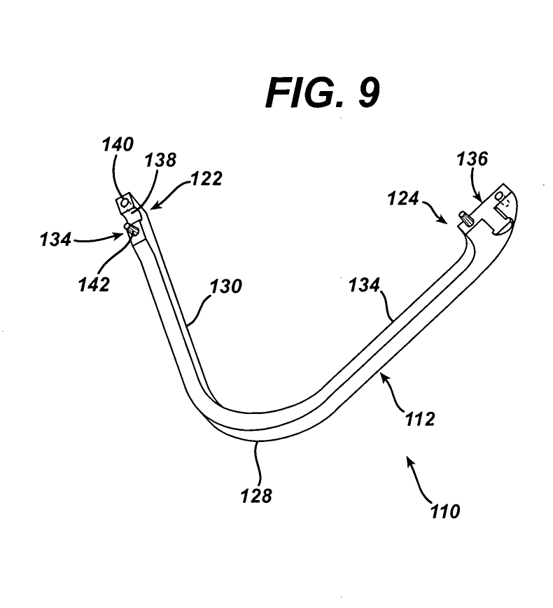

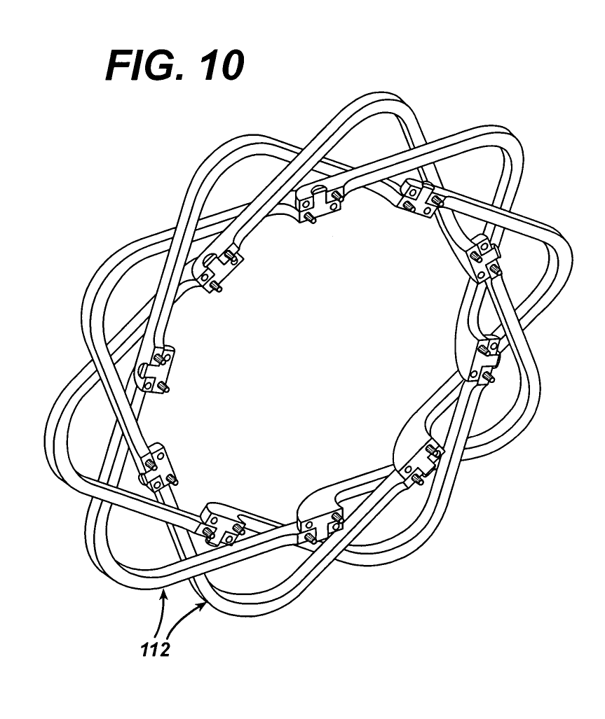

For instance, in FIGS. 8-10, another anastomotic ring device 110 is formed from molded arcuate members 112 that show further advantages of forming two crowns 114, 116 with attachments 120 at a circular midpoint 118. With particular reference to FIG. 9, each arcuate member 112 has a first end 122 and a second end 124 convergingly joined at a curved point 128. Each first end 122, 124 bends perpendicularly to their respective elongate shafts 130, 132 presenting respectively pin hinge receiving surface 134 and a pin hinge surface 136. The pin hinge receiving surface 134 includes a lateral half cylinder recess 138 interposed between a distally presented female attachment feature 140 and a proximally disposed male attachment feature 142. The pin hinge surface 136 includes a half pin 144 interposed between a distally disposed female attachment feature 146 and a proximally disposed male attachment feature 148.

These first and second ends 122, 124 of the arcuate member 112 in one crown 114 facilitate a rigid attachment at attachment 120 to rotated identical arcuate members of the other crown 116. The joined first ends 122 between the two arcuate members 112 forms a through hole of the two half cylinder recesses 138 that receive a pin hinge formed from two half pins 144 formed from two second ends 124. Thus each arcuate member 112 is interwoven with its two adjacent arcuate members 112, moves in concert with its two attached arcuate members 112 in the other crown 116 and is hingedly connected to arcuate members 112 that are on the other side to the adjacent arcuate members 112.

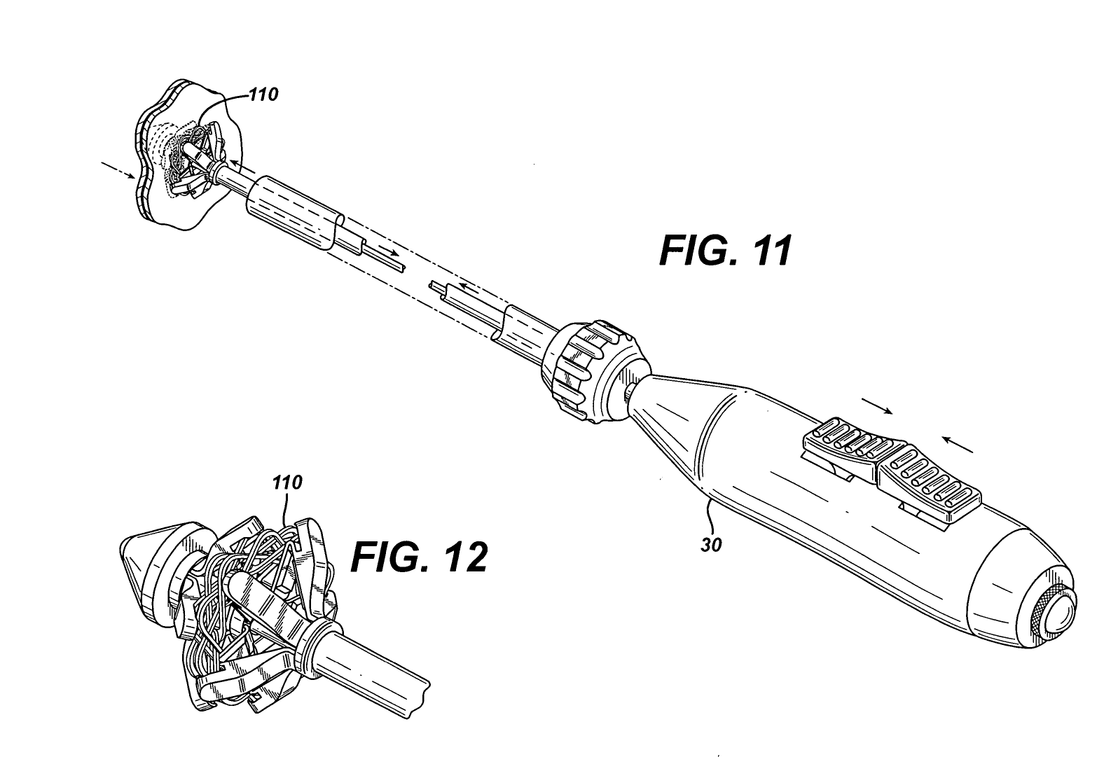

Sufficient friction exists in the hinged connection between arcuate members at the midpoint 118 that when placed in position, such as depicted in FIGS. 11-12 by an applier 30, the anastomotic ring device 110 tends to stay in its actuated position. Alternatively, the anastomotic ring device 110 is intended to maintain the anastomotic opening and requires a secondary fastening element to remain in the actuated position, such as sutures fastening arcuate members 112 to the tissue and to one another.

Although such molded arcuate members 112 may be assembled in an unactuated, cylindrical fashion as previously described above for the wire anastomotic ring device 10, in FIG. 9, it is shown how one crown 114 may be formed in an actuated configuration, readily prepared to accept individual arcuate members 112 of the other crown or a fully assembled bottom crown 116.

Each molded arcuate member may be formed from a bioabsorbable material, such as a biofragmentable polymer mixture that eventually passes out of the digestive tract.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art.

For example, a molded arcuate member consistent with aspects of the invention may form a hinged relationship rather than a rigid attachment to respective arcuate members of the inverted crown.

As a further example, an anastomosis ring device may include a circular fixture or band at its midpoint for attaching the arcuate members that remains part of the anastomosis ring device, intended to sit at a tissue juncture of the anastomosis.

1. An anastomosis device, comprising:
a first plurality of arcuate members arranged in a first position in a cylindrical crown shape with each arcuate member having legs overlapping at least one adjacent arcuate member; and
a second plurality of arcuate members arranged in a first position in an inverted cylindrical crown shape with each arcuate member having legs overlapping at least one adjacent arcuate member of the second plurality and connected to a leg of an arcuate member of the first plurality;
wherein the woven tube thus formed is operably configured to transform into a second position comprising a hollow rivet shape with each arcuate member outwardly deflected from a longitudinal axis of its respective cylindrical crown toward apposing arcuate members of the other cylindrical crown.

2. The anastomosis device of claim 1, wherein the arcuate members comprise a shape memory effect alloy.

3. The anastomosis device of claim 1, wherein the legs of the arcuate members of the first plurality are attached to the respective arcuate member of the second plurality by a connecting member.

4. The anastomosis device of claim 3, wherein the connecting members comprises a selected one of a group consisting of a snap fit, a glue, an ultrasonically welded portion, and a thermally melted polymer.

5. The anastomosis device of claim 3, wherein the legs of the arcuate members of the first plurality are attached to the respective arcuate member of the second plurality by a rigid connecting member, a petal formed by the first arcuate member actuating generally in a plane with the respective attached arcuate members pivoting about a cylindrical midpoint of the anastomosis device.

6. The anastomosis device of claim 5, wherein each leg is further hingedly coupled at the circular midpoint of the anastomosis device to at least one other leg.

7. The anastomosis device of claim 3, wherein the legs of the arcuate members of the first plurality are attached to the respective arcuate member of the second plurality by a pivotal connecting member.

8. The anastomosis device of claim 3, wherein the connecting member comprises a band at a midpoint of the device connected to each arcuate member.

9. An anastomosis ring device, comprising:
a means for forming a half cylindrical which actuates into a first apposing member about a midpoint at an anastomosis tissue juncture; and
a means for forming an inverted half cylinder which actuates into a second apposing member at the midpoint at the anastomosis tissue juncture.

10. An anastomosis ring device, comprising a plurality of arcuate members operably configured to be arranged into two crowns attached to one another to present petal circumferentially hinged at a circular midpoint, each arcuate member comprising a pair of diverging connected legs.

11. The anastomosis ring device of claim 10, wherein each arcuate member further comprises:
a first end having a pin receiving recess; and
a second end having a pivot pin; wherein the first end of a first arcuate member pivotally connects to a second end of a second arcuate member and the second end of the first arcuate member connects to a first end of a third arcuate member.

12. The anastomosis ring device of claim 11, wherein the first, second and third arcuate members reside within the same crown, the first and second ends of each arcuate member rigidly connected to a respective first and second end of a fourth and fifth inverted arcuate member of the crown.

13. The anastomosis ring device of claim 12, wherein the first and second ends are aligned parallel to one another and perpendicularly oriented with respect to a plane formed by the diverging connection of the two legs.
The applier of claim 1, wherein the actuating member is engaged to the distal ring.
